(19) United States
(12) Patent Application Publication
(10) Pub. No.: US 2020/0088712 A1
(43) Pub. Date: Mar. 19, 2020

(54) METHOD FOR DETECTING JOINT DISEASES

(71) Applicants: ECOLE CENTRALE DE LYON, ECULLY (FR); CENTRE NATIONAL DE LA RECHERCHE SCIENTIFIQUE (CNRS), PARIS (FR); ECOLE NATIONALE D'INGENIEURS DE SAINT ETIENNE, SAINT ETIENNE (FR); INSTITUT ENSEIGNEMENT SUPERIEUR ET RECHERCHE EN ALIMENTATION, SANTE ANIMALE, AGRONOMIQUES ET, MARCY L'ETOILE (FR)

(72) Inventors: Catherine BOSSER, CHARBONNIERES LES BAINS (FR); Thierry HOC, CHARBONNIERES LES BAINS (FR); Caroline BOULOCHER, LA TOUR DE SALVAGNY (FR)

(73) Assignees: ECOLE CENTRALE DE LYON, ECULLY (FR); CENTRE NATIONAL DE LA RECHERCHE SCIENTIFIQUE (CNRS), PARIS (FR); ECOLE NATIONALE D'INGENIEURS DE SAINT ETIENNE, SAINT ETIENNE (FR); INSTITUTENSEIGNEMENT SUPERIEUR ET RECHERCHE EN ALIMENATION, SANTE ANIMALE, AGRONOMIQUES ET, MARCY L'ETOILE (FR)

(21) Appl. No.: 16/466,877

(22) PCT Filed: Nov. 22, 2017
(86) PCT No.: PCT/EP2017/080088

§ 371 Date: Jun. 5, 2019

(30) Foreign Application Priority Data
Dec. 5, 2016 (FR) 1661956

(51) Int. Cl. G01N 33/487; G01N 21/65; G01N 1/28

(52) U.S. Cl. CPC G01N 33/487; G01N 1/28; G01N 21/65

(57) ABSTRACT

The present invention concerns an in vitro process for diagnosing joint disease in a mammal and/or determining the type of joint disease and/or determining the stage of said disease and/or predicting the course of said disease, comprising the following steps:
a) depositing a drop of a synovial fluid sample from said mammal onto a flat substrate made of an inorganic material, such as glass;
b) drying the drop deposited in step a);
c) measuring at least one parameter indicative of the morphological features of the drop dried in step b), whereby a value for said parameter is obtained, and
d) comparing each value measured in step c) with a reference value representative of a reference synovial fluid,
characterized in that said parameter is selected from (i) the maximum height (H) of the bead formed on the surface of the drop, (ii) the area of the drop and (iii) the surface profile (Z) of the drop.

The present invention concerns a diagnostic process.

More specifically, the present invention concerns a process for diagnosing, determining the type or assessing the severity of joint disease affecting a mammal, in particular osteoarthritis.

Joint diseases, or arthropathies, affect the joints and include osteoarthritis, arthritis, rheumatoid arthritis, ankylosing spondylitis and arthralgia.

Among these joint diseases, osteoarthritis, also known as arthrosis, is the most common. The prevalence of osteoarthritis is constantly increasing worldwide, as it is linked to an aging and overweight population. It manifests most often as mechanical pain and/or discomfort during joint movements.

Many joints can be affected by osteoarthritis. The most affected are the cervical and lumbar spine (70-75%), the knee (40%), the thumb (30%), the hip and ankle (10%) and the shoulders (2%).

Osteoarthritis has long been presented as “wear and tear” of the cartilage, whereas it is a destructive and inflammatory syndrome, associated with various risk factors. Scientists no longer speak of osteoarthritis in general but of types of osteoarthritis according to the patient's profile: age-related osteoarthritis, obesity-related osteoarthritis, joint disease-related osteoarthritis, etc. The current objective is to individualize management and treatment according to these different profiles.

Cartilage lesions do not regress over time and their progression is not linear. It can be very rapid and require the implantation of a prosthesis within 5 years. Osteoarthritis can also progress slowly, over several years, without inducing major disability.

The diagnosis of the disease is usually established by (i) the patient's own observation of pain or discomfort in a joint, and (ii) an X-ray of the joint in question to visually determine the state of cartilage degradation. However, there is no relationship between the extent of radiological lesions and the severity of osteoarthritis pain.

Clinical diagnostic criteria have been developed by the American College of Rheumatology (ACR), based on the description of pain and/or discomfort (Litwic et al., 2013).

X-rays can show very characteristic signs of osteoarthritis such as:
joint space narrowing,
osteophytes, i.e. a surplus of bone around the joint,
subchondral sclerosis.
subchondral bone cysts, which may be compared to “holes”.

The severity of the disease is most commonly measured using the scale of Kellgren & Lawrence, who established a K/L radiological severity score ranging from 0 to 4, this scale having now been adopted by the WHO.

To date, there are only symptomatic treatments to relieve pain. No osteoarthritis therapy capable of protecting cartilage is currently available.

Joint disease research is currently focused on two main objectives, finding therapeutic targets and identifying biomarkers (i) to diagnose the disease before disabling symptoms appear, (ii) to determine the type of disease in order to personalize treatment, (iii) to determine the stage of the disease, and (iv) to predict the course of the disease.

The present invention concerns the achievement of this second objective.

Currently, and as mentioned above, diagnosis is based on clinical examination and radiological images. Researchers are working to identify molecules whose presence or concentration in serum or urine could be used as biomarkers, but at present there is no candidate molecule to categorize patients (Lotz et al., 2013). However, it has been shown that there is a relationship between the presence of certain circulating microRNAs in the serum and the severity of knee and hip osteoarthritis (Beyer et al., 2015)

During a flare-up with synovial fluid effusion, the analysis of this fluid collected during joint puncture can help confirm and/or refine the diagnosis.

In vitro processes to assess the severity of joint disease in a mammal, based on synovial fluid samples, are actively sought.

The article (Esmonde-White et al., 2009a) describes in particular a process for discriminating different stages of the disease, consisting of measuring, on a dried synovial fluid drop, the following parameters: presence of crystals in the drop center, and presence of radial erosion marks at the drop edge.

In addition, by analyzing the composition of the pathological synovial fluid by Raman spectroscopy, Dr. Esmonde-White's team identified a relationship between said Raman spectrum of the synovial fluid and the severity, according to the K/L score, of knee osteoarthritis. In particular, Raman band intensity ratios have been significantly correlated with the radiographic severity of the disease (Esmonde-White et al., 2009b).

In the application US 2007/0082409, Raman spectrometric analysis of synovial fluid was used to determine the presence of certain molecules, including hyaluronic acid (HA). This joint lubricant is a biomarker of joint diseases: in these pathologies, it is either degraded or present in lower concentrations due to the infiltration of fluids and proteins into the joint; moreover, the hyaluronic acid present is in a different molecular form, of lower molecular weight, this form not having the same viscoelastic properties as the hyaluronic acid present in healthy joints.

The detection of this biomarker is indicative of the stage of the disease, but remains difficult to implement, as Raman spectroscopy equipment is required.

In the case of joint diseases, other molecular changes in the synovial fluid are observed, including an increase in protein concentration and modification of the level of glycosaminoglycans (GAGs): increase in their concentration in the early stages, decrease in the late stages.

The inventors have demonstrated a relationship between the morphological features of a synovial fluid drop and the presence, type or stage of joint disease affecting the mammal from which the synovial fluid is obtained.

The inventors have also demonstrated a correlation between the morphological features of a synovial fluid drop and the hyaluronic acid and protein content of said synovial fluid.

The process as presented below makes it possible to diagnose joint disease and/or obtain information on its type or stage and/or predict its course, in a simple, rapid and low-cost manner.

### SUMMARY OF THE INVENTION

The present invention concerns an in vitro process for diagnosing joint disease in a mammal and/or determining the type of joint disease and/or determining the stage of said disease and/or predicting the course of said disease, comprising the following steps:

a) depositing a drop of a synovial fluid sample from said mammal onto a flat substrate made of an inorganic material, such as glass;

b) drying the drop deposited in step a);

c) measuring at least one parameter indicative of the morphological features of the drop dried in step b), whereby a value for said parameter is obtained, and

d) comparing each value measured in step c) with a reference value representative of a reference synovial fluid,

characterized in that said parameter is selected from (i) the maximum height (H) of the bead formed on the surface of the drop, (ii) the area of the drop and (iii) the surface profile (Z) of the drop.

In addition, according to a particular implementation, the process may also include the following steps:

e) determining the Raman spectrum of the drop dried in step b), and

f) comparing the Raman spectrum determined in step e) with a Raman spectrum representative of a reference synovial fluid.

The present invention also concerns an in vitro process for monitoring a mammal with joint disease, comprising the following steps:

a) depositing a drop of a synovial fluid sample from said mammal, obtained at a time T₁, onto a flat substrate made of an inorganic material, such as glass;

b) drying the drop deposited in step a);

c) measuring at least one parameter indicative of the morphological features of the drop dried in step b), whereby a value for said parameter is obtained, and

d) comparing each value measured in step c) with a value obtained by applying steps (a) to (c) of the process to a synovial fluid sample from said mammal obtained at a time T₀ prior to time T₁,

characterized in that said parameter is selected from (i) the maximum height (H) of the bead formed on the surface of the drop, (ii) the area of the drop and (iii) the surface profile (Z) of the drop.

The present invention also concerns an in vitro process for determining the efficacy of a treatment for joint disease in a mammal with said disease to which said treatment is administered, comprising the following steps:

a) depositing a drop of a synovial fluid sample from said mammal, obtained after the start of said treatment, onto a flat substrate made of an inorganic material, such as glass;

b) drying the drop deposited in step a);

c) measuring at least one parameter indicative of the morphological features of the drop dried in step b), whereby a value for said parameter is obtained, and

d) comparing each value measured in step c) with a reference value representative of a reference synovial fluid,

characterized in that said parameter is selected from (i) the maximum height (H) of the bead formed on the surface of the drop, (ii) the area of the drop and (iii) the surface profile (Z) of the drop.

The present invention also concerns a process for in vivo screening a non-human mammal for candidate compounds intended to treat at least one joint disease, comprising the following steps:

a) depositing a drop of a synovial fluid sample from said mammal, obtained after the start of administration of one of said candidate compounds to said non-human mammal, onto a flat substrate made of an inorganic material, such as glass;

b) drying the drop deposited in step a);

c) measuring at least one parameter indicative of the morphological features of the drop dried in step b), whereby a value for said parameter is obtained, and

d) comparing each value measured in step c) with a reference value representative of a reference synovial fluid,

characterized in that said parameter is selected from (i) the maximum height (H) of the bead formed on the surface of the drop, (ii) the area of the drop and (iii) the surface profile (Z) of the drop.

The present invention also concerns a process for the molecular characterization of a mammalian synovial fluid, comprising the following steps:

a. depositing a drop of a synovial fluid sample from said mammal onto a flat substrate made of an inorganic material, such as glass;

b. drying the drop deposited in step a);

c. measuring:
the area of the drop dried in step b), whereby a value (A) for this area is obtained, and/or
the maximum height (H) of the bead formed on the surface of the drop dried in step b), whereby a value (H) for said height is obtained, and

d. comparing these values (A) and/or (H) with the following ranges of values:
the value (A) is compared with a range of values correlated with the concentration of hyaluronic acid present in a synovial fluid; and
the value (H) is compared with a range of values correlated with the concentration of proteins present in a synovial fluid.

### FIGURES

FIG. 1: Schematic representation of the drop deposition protocol (DDRS).

FIG. 2: Images obtained by the DDRS technique:

(a) White-light microscope images of a dry synovial fluid (SF) drop showing typical structures in two distinct regions: a translucent peripheral area with radial cracks and a central area with crystalline deposits of dendritic shape. Raman acquisitions were made in the peripheral cardinal areas and are represented by rectangles.

(b) The upper image measured with an interferometer shows that the four regions of interest (ROI) are positioned in areas where solutes are concentrated.

FIG. 3. Examples of 2D and 3D images for a healthy SF (upper part of the figure) and an osteoarthritic SF (OA SF—lower part of the figure): the healthy SF drop is smaller in size than the OA SF drop; on the healthy SF drop, a black circle is visible between the outer periphery and the center, and the maximum height of the bead is about 12 μm (height is represented by black/white color variations); on the OA SF drop, there is clearly a peripheral bead, the maximum height of which is about 25 μm and which is wider than on the healthy SF drop.

FIG. 4. A) Box plots (representing medians, quartiles and minimum and maximum values) of drop areas (left part of FIG. 4A) and bead heights between drops (right part of FIG. 4A) of healthy and osteoarthritic SF: stars indicate significant differences* (p<0.05) and ** (p<0.01).

B) Z-profiles along the periphery of the drops on healthy and osteoarthritic SF: the bold lines and shaded areas represent the means and standard deviations, respectively. The upper curve illustrates the results obtained on drops from OA subjects. The lower curve shows the results obtained on drops from healthy subjects.

FIG. 5. (A) Normalized mean Raman spectra of healthy SF in dogs (n=6); macroscopically all samples had a normal appearance (viscosity, turbidity, transparency, color). Y-axis: signal intensity, expressed in arbitrary units (a.u.). X-axis: wavenumbers, expressed in cm⁻¹.

(B) Normalized Raman spectrum of osteoarthritic SF in dogs (n=6); samples were relatively inflammatory with a color ranging from straw yellow to orange. The spectrum in bold represents the healthy SF Raman spectral fingerprint, the gray area corresponds to the standard deviation. Y-axis: signal intensity, expressed in arbitrary units (a.u.). X-axis: wavenumbers, expressed in cm⁻¹.

(C) Spectral regions of interest.

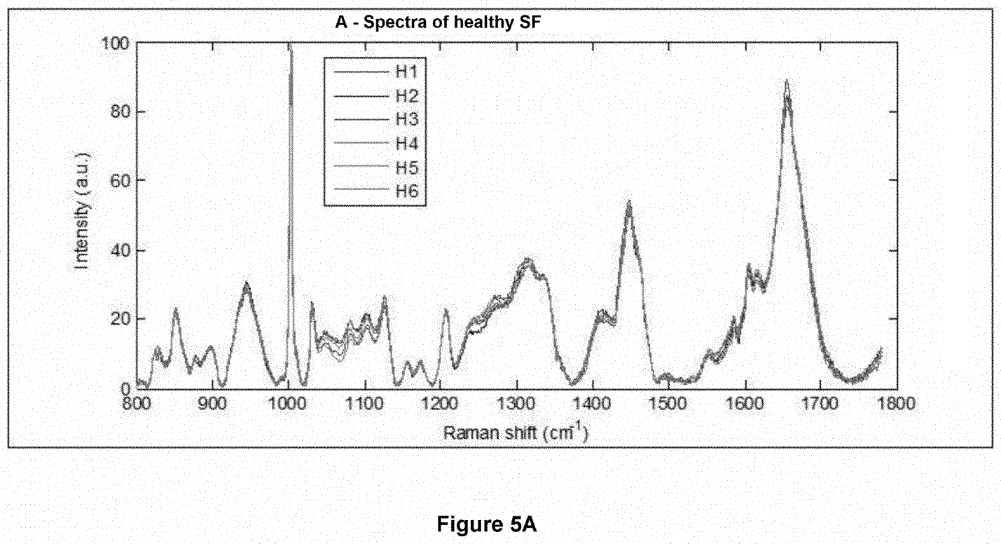

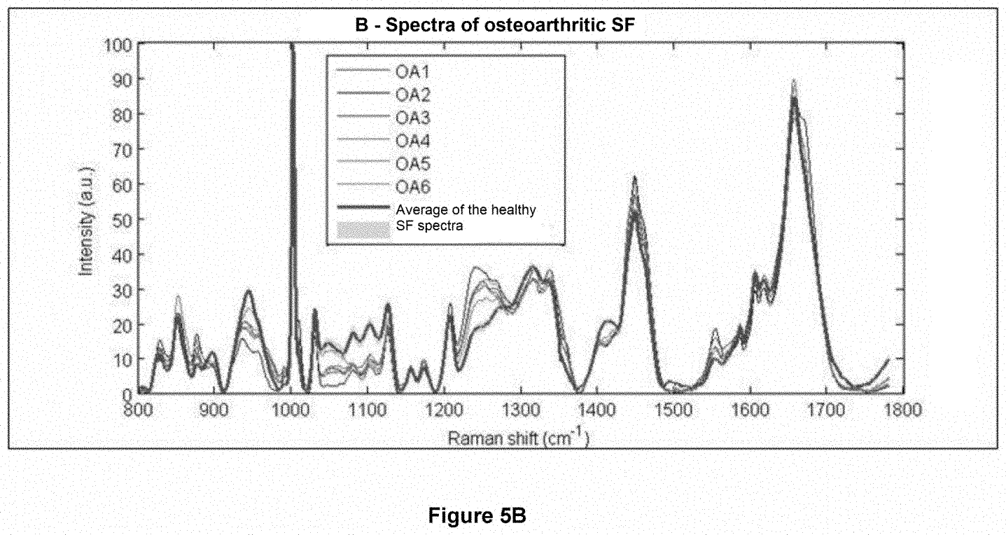

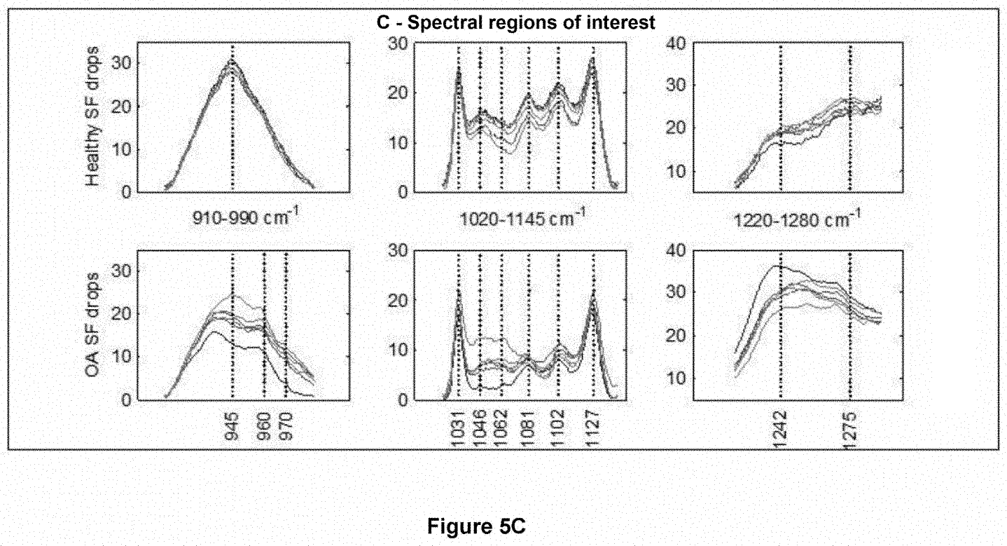

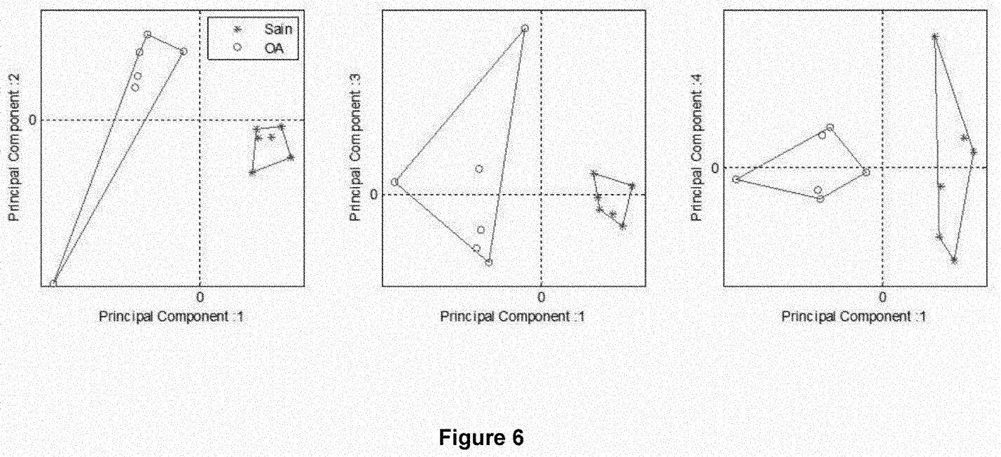

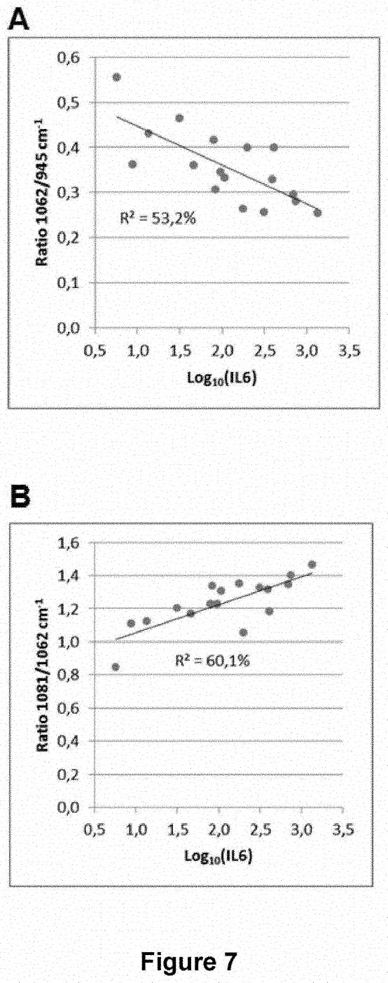

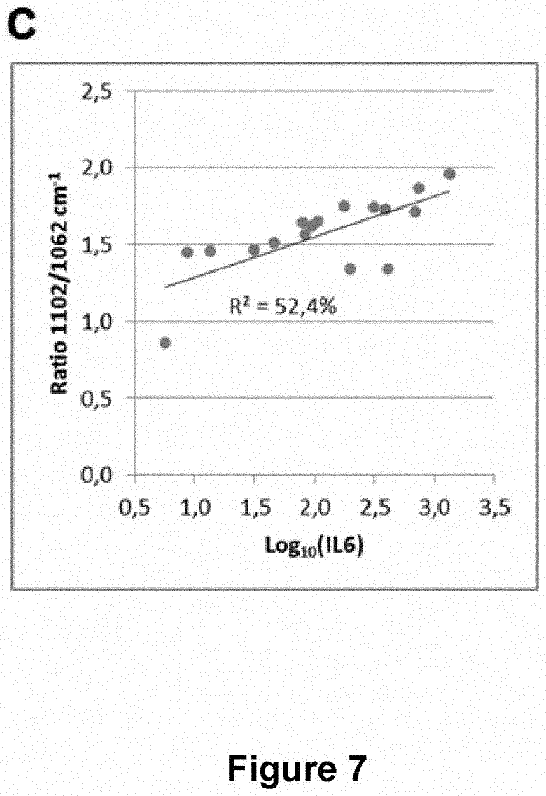

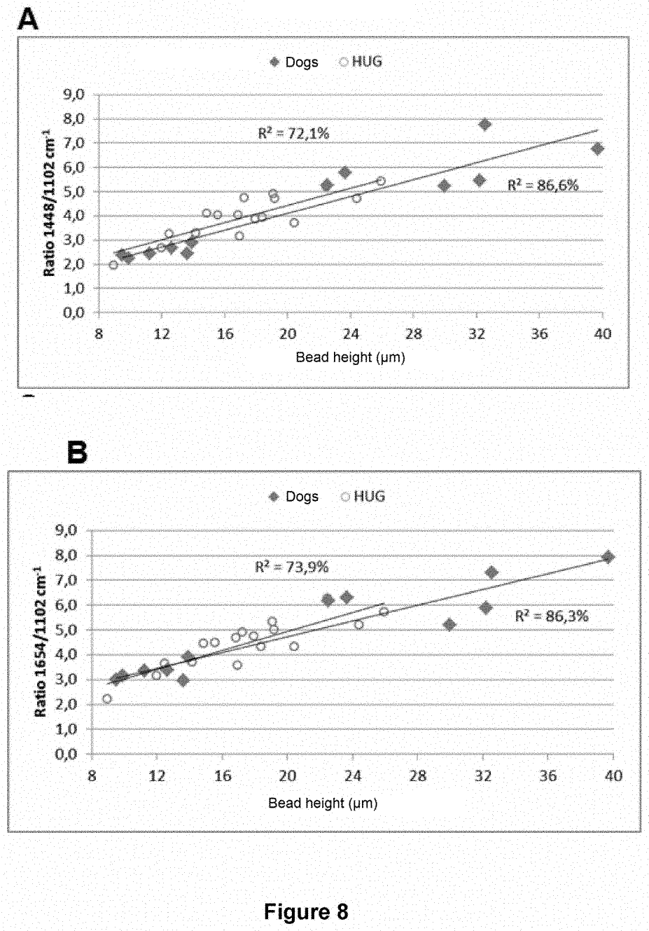

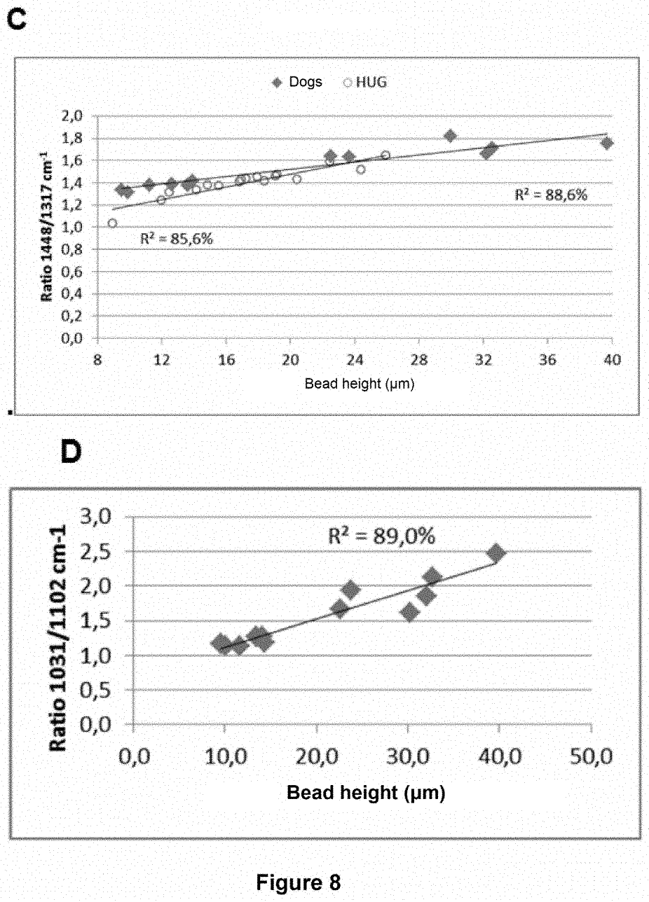

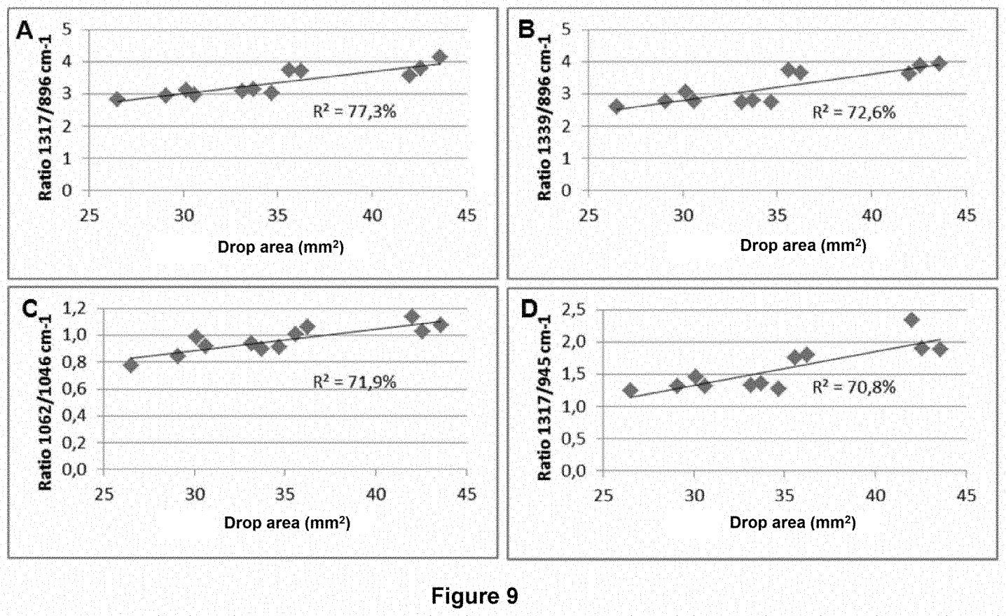

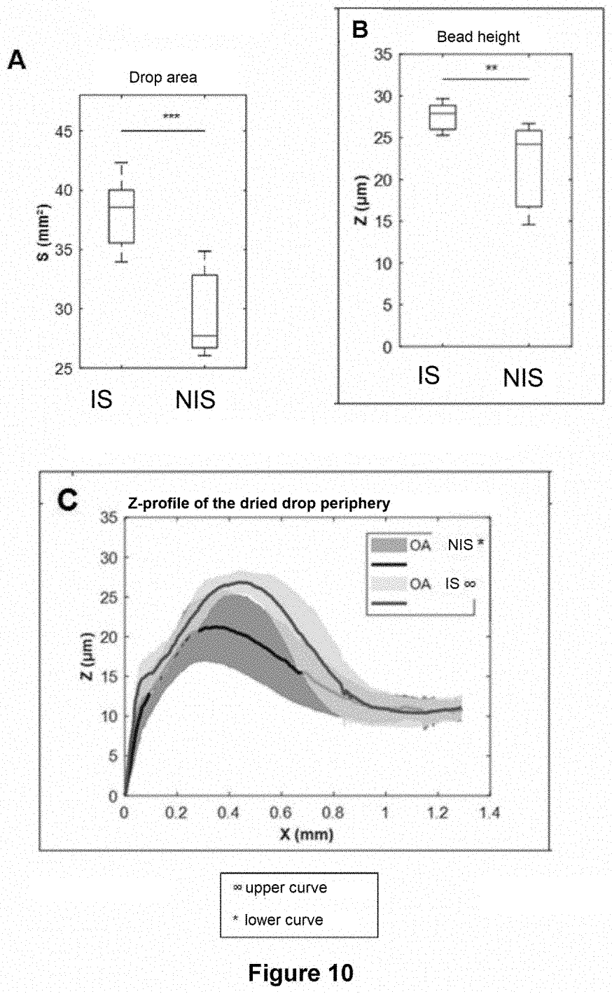

Upper part of FIG. 5C: spectra obtained from drops from healthy subjects; From left to right: (i) spectra in the spectral range 910 to 990 cm⁻¹; (ii) spectra in the spectral range 1020 to 1145 cm⁻¹; (iii) spectrum in the spectral range 1220 to 1280 cm⁻¹;

Lower part of FIG. 5C: spectra obtained from drops from OA subjects; From left to right: (i) spectra in the spectral range 910 to 990 cm⁻¹; (ii) spectra in the spectral range 1020 to 1145 cm⁻¹; (iii) spectra in the spectral range 1220 to 1280 cm⁻¹;

The vertical lines in FIG. 5C correspond to the wavelengths specified on the y-axis of the lower part of the figure, respectively from left to right: 945 cm⁻¹, 960 cm⁻¹, 970 cm 1031 cm⁻¹, 1046 cm⁻¹, 1062 cm⁻¹, 1081 cm⁻¹, 1102 cm⁻¹, 1127 cm⁻¹, 1242 cm⁻¹ and 1275 cm⁻¹.

FIG. 6. Results of a principal component analysis (PCA) based on 210 Raman spectrum ratios.

Each point (star for healthy samples, circle for OA samples) represents the 210 ratios of the Raman spectrum of a sample. The groups of healthy and OA points are clearly located in separate regions.

FIG. 7. This figure shows a good correlation between three ratios of Raman spectrum peaks and the amount of interleukin 6 (IL6) in human SF samples.

(A) Y-axis: the signal ratio of the peak at 1062 cm to the peak at 945 cm⁻¹. X-axis: the amount of IL6, expressed as log₁₀ concentration.

(B) Y-axis: the signal ratio of the peak at 1081 cm⁻¹ to the peak at 1062 cm⁻¹. X-axis: the amount of IL6, expressed as log₁₀ concentration.

(C) Y-axis: the signal ratio of the peak at 1102 cm⁻¹ to the peak at 1062 cm⁻¹. X-axis: the amount of IL6, expressed as log₁₀ concentration.

FIG. 8. The three Raman peak ratios 1448/1102 cm⁻¹ (A); 1654/1102 cm⁻¹ (B); and 1448/1317 cm⁻¹ (C) are correlated with height value (H) of the beads, in both human and dog SF samples. The 1031/1102 cm⁻¹ ratio (D) also associated with proteins is highly correlated (R²>85%) with bead height value (H) in dogs.

FIG. 9. The four Raman peak ratios in relation to hyaluronic acid: 1317/896 cm⁻¹ (A); 1339/896 cm⁻¹ (B); 1062/1046 cm⁻¹ (C) and 1317/945 cm⁻¹ (D), are correlated with the area value (A) of the synovial fluid drop, in SF samples from dogs.

FIG. 10. (A) Box plot (representing medians, quartiles and minimum and maximum values) of drop surfaces between IS (inflammatory stage) and NIS (non-inflammatory stage) SF drops: stars indicate significant differences* (p<0.05) and ** (p<0.01).

(b) Box plot (representing medians, quartiles and minimum and maximum values) of bead heights between IS (inflammatory stage) and NIS (non-inflammatory stage) SF drops: stars indicate significant differences* (p<0.05) and ** (p<0.01).

(C) Z-profiles along the periphery of drops on IS and NIS SF: the bold lines and shaded areas represent the means and standard deviations, respectively. The upper curve illustrates the results obtained on drops from IS subjects. The lower curve shows the results obtained on drops from NIS subjects.

### DETAILED DESCRIPTION OF THE INVENTION

The inventors have identified new biomarkers to diagnose or determine the type or severity of joint disease, these biomarkers being parameters indicative of the morphological features of a dried drop of synovial fluid derived from a mammal.

Joint disease, for the purposes of the invention, means a disease affecting the joints and in particular the cartilage present in these joints. This joint disease can be inflammatory or not, degenerative or not, and is notably selected from osteoarthritis, arthritis, rheumatoid arthritis, ankylosing spondylitis and arthralgia.

Mammal, for the purposes of the invention, means in particular domestic mammalian animals such as cats, dogs, hamsters, rabbits, guinea pigs and ferrets; farmed animals such as sheep, cattle, goats, horses, camelids and deer are also concerned by the present invention.

More specifically, the present invention concerns joint diseases affecting human beings. In the present application, the term “patient” refers to a human being, child or adult, affected by joint disease.

According to a first aspect, the present invention concerns an in vitro process for diagnosing joint disease in a mammal, comprising the following steps:

a) depositing a drop of a synovial fluid sample from said mammal onto a flat substrate made of an inorganic material, such as glass;

b) drying the drop deposited in step a);

c) measuring at least one parameter indicative of the morphological features of the drop dried in step b), whereby a value for said parameter is obtained, and

d) comparing each value measured in step c) with a reference value representative of a reference synovial fluid,

characterized in that said parameter is selected from (i) the maximum height (H) of the bead formed on the surface of the drop, (ii) the area of the drop and (iii) the surface profile (Z) of the drop.

To “diagnose” means, for the purposes of the invention, to establish the presence of joint disease in a patient, including at a very early stage of the disease when clinical symptoms are difficult to detect or interpret.

According to a second aspect, the present invention concerns an in vitro process for determining the type of joint disease affecting a mammal, comprising the following steps:

a) depositing a drop of a synovial fluid sample from said mammal onto a flat substrate made of an inorganic material, such as glass;

b) drying the drop deposited in step a);

c) measuring at least one parameter indicative of the morphological features of the drop dried in step b), whereby a value for said parameter is obtained, and

d) comparing each value measured in step c) with a reference value representative of a reference synovial fluid,

characterized in that said parameter is selected from (i) the maximum height (H) of the bead formed on the surface of the drop, (ii) the area of the drop and (iii) the surface profile (Z) of the drop.

To “determine the type of joint disease” means, for the purposes of the invention, to determine the origin(s) of the joint disease, and in particular to determine whether it is an inflammatory disease or not, or whether it is a disease related to the patient's profile (age, overweight) or to the presence of a pathogen (bacteria, viruses), or whether it is a degenerative disease.

In particular, it will be possible to determine which disease affects the patient, among the following joint diseases: osteoarthritis, arthritis, rheumatoid arthritis, ankylosing spondylitis and arthralgia.

According to a third aspect, the present invention concerns an in vitro process for evaluating the stage of joint disease affecting a mammal, comprising the following steps:

a) depositing a drop of a synovial fluid sample from said mammal onto a flat substrate made of an inorganic material, such as glass;

b) drying the drop deposited in step a);

c) measuring at least one parameter indicative of the morphological features of the drop dried in step b), whereby a value for said parameter is obtained, and

d) comparing each value measured in step c) with a reference value representative of a reference synovial fluid,

characterized in that said parameter is selected from (i) the maximum height (H) of the bead formed on the surface of the drop, (ii) the area of the drop and (iii) the surface profile (Z) of the drop.

To “evaluate the stage of joint disease”, also referred to in the present application as “evaluate the severity of joint disease”, for the purposes of the present invention, means to determine the stage of the disease according to previously established clinical and biological scores, such as the K/L radiological severity score.

This process makes it possible to distinguish between the inflammatory and non-inflammatory stages of joint disease.

According to one implementation of the invention, the process for determining the stage of joint disease affecting a mammal also makes it possible to give a prognosis of the course of the disease.

According to a fourth aspect, the present invention concerns an in vitro process for predicting the progression of joint disease, comprising the following steps:

a) depositing a drop of a synovial fluid sample from said mammal onto a flat substrate made of an inorganic material, such as glass;

b) drying the drop deposited in step a);

c) measuring at least one parameter indicative of the morphological features of the drop dried in step b), whereby a value for said parameter is obtained, and

d) comparing each value measured in step c) with a reference value representative of a reference synovial fluid,

characterized in that said parameter is selected from (i) the maximum height (H) of the bead formed on the surface of the drop, (ii) the area of the drop and (iii) the surface profile (Z) of the drop.

According to a fifth aspect, the present invention concerns an in vitro process for diagnosing joint disease in a mammal and the severity of the joint disease, comprising steps a) to d) listed above.

According to a sixth aspect, the present invention concerns an in vitro process for diagnosing joint disease in a mammal and determining the type of said joint disease, comprising steps a) to d) listed above.

According to a seventh aspect, the present invention concerns an in vitro process for assessing the severity and determining the type of joint disease affecting a mammal, comprising steps a) to d) listed above.

According to an eighth aspect, the present invention concerns an in vitro process for diagnosing joint disease in a mammal, for assessing the severity and determining the type of said disease, comprising steps a) to d) listed above.

The processes according to the invention all comprise four successive steps a) to d), the implementation of which is detailed below.

Step a)

The first step consists of depositing a drop of a synovial fluid sample from said mammal onto a flat substrate made of an inorganic material, such as glass.

Synovial Fluid Sample

Synovial fluid (SF), or synovia, is a biological liquid produced by the synovial membrane. This liquid is viscous and transparent or pale yellow. It consists of a dialysate of serum containing electrolytes, glucose, proteins, glycoproteins and hyaluronic acid, and filtered interstitial fluid from blood plasma.

The presence of this liquid in the joints reduces friction through its lubricity, absorbs shock, provides oxygen and nutrients to joint cartilage chondrocytes, and removes carbon dioxide and metabolic waste from these cells.

The synovial fluid samples tested according to the process of the invention are collected from mammalian joints affected or likely to be affected by joint disease. Samples are collected sterile by surgical intervention. The samples are then frozen for storage.

Before the drop is deposited, these samples are centrifuged to remove the cells present in the synovial fluid.

These SF samples can be diluted with conventional buffers or undiluted. They can also be treated with proteases and RNases in order to best preserve the biological constituents.

According to a preferred aspect of the invention, the deposited drop comes from an undiluted SF sample. According to another aspect, the SF sample is not treated with proteases and/or RNases.

“Drop Deposition” Technique

The drop deposition (DD) technique is well suited for the examination of low abundance biofluids, such as tears, synovial fluid or cerebrospinal fluid, as there is often not enough fluid to perform chromatographic or conventional electrophoresis analyses. Biofluids can be prepared using a single drop of liquid, and the same dried drop can be examined using multiple techniques such as optical microscopy, white-light interferometry, atomic force microscopy (AFM), matrix-assisted laser desorption/ionization (MALDI), mass spectrometry, acoustic-mechanical impedance or optical spectroscopy, notably Raman spectroscopy.

The multiple data obtained from a few microliters of biofluid are representative of the physical and chemical properties of the biological fluid analyzed.

Nature of the Material Constituting the Flat Surface

In the process according to the invention, the deposition of a drop of a synovial fluid sample is carried out on a flat substrate made of an inorganic material, in particular of a water-repellent nature.

The term “material of water-repellent nature” refers to an impermeable material that repels water, the latter being unable penetrate the pores of the material due to the very nature or the coating of the material.

According to a particular implementation of the invention, the flat substrate is pre-treated to remove any trace of grease from its surface.

According to a particular implementation of the invention, the flat substrate has a water contact angle comprised between 50° and 90°.

According to a particular implementation of the invention, the flat substrate is transparent. According to a preferred implementation of the invention, the flat substrate is made of glass.

For the purposes of the invention, the term “glass” refers to amorphous solids obtained by heating a mixture, in appropriate proportions, of silica and metal oxides. They are therefore mixed silicates, solid, non-crystalline, transparent and fragile, formed by the disordered juxtaposition of silica SiO₄ tetrahedra and by the presence of alkaline, alkaline earth oxides, lead, aluminum, etc. Glass is hard, brittle and transparent to visible light. The glass considered in the present invention is a mineral glass, not an organic glass.

It may be soda-lime glass, or borosilicate glass, of a quality suitable for microscopy.

The flat substrate is in particular a microscope slide made of optical quality, thin (about 1 mm), uncoated glass. This substrate is particularly advantageous due to its very low cost and wide availability.

The article by Esmonde-White et al., 2014, presents a study on the DD technique, based on two biofluids: plasma and synovial fluid, where several slightly hydrophilic materials were tested as a substrate for drop deposition. These include in particular glass coated with gold or calcium fluoride (CaF₂). These materials are used because they optimize the reading of the Raman spectrum, the background being much lower than that generated by uncoated glass. However, on a flat substrate made of these materials, the authors of the article did not observe any differences in the morphological features of SF drops from patients with osteoarthritis, at different stages of the disease.

Step b): Drying the Drop Deposited in Step a);

Drying will be carried out in the most appropriate manner, as easily determined by the skilled person.

According to one implementation of the invention, drying of the synovial fluid drop is carried out at room temperature, for at least 8 hours, or at least 12 hours, or at least 24 hours.

According to another implementation of the invention, drying of the drop is carried out for 30 minutes or one hour, at 37° C.

According to other implementations of the invention, the drying time may be shortened to less than 30 minutes. Indeed, the diagnostic process according to the invention will preferably be implemented in a rapid manner, in order to obtain a diagnosis as soon as possible.

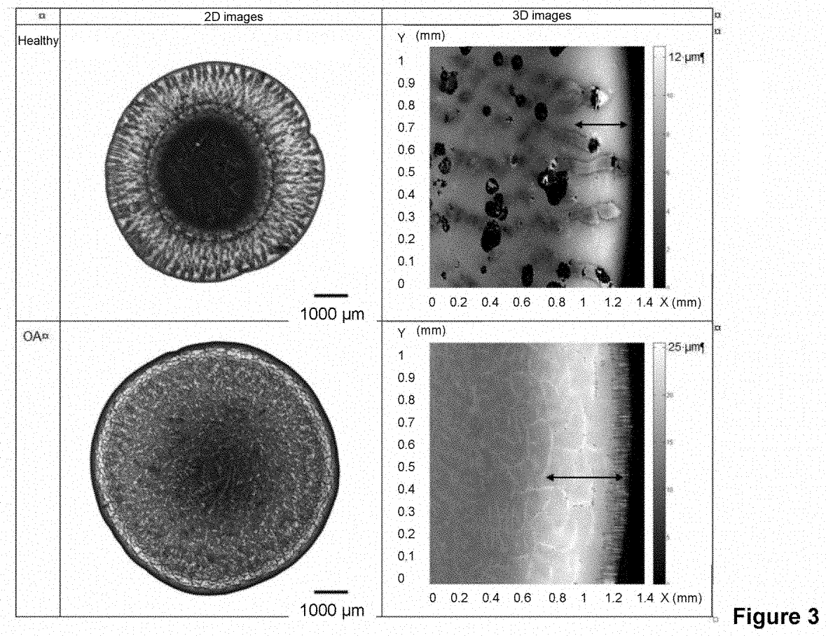

Once the drop has dried, 2- and 3-dimensional images can be obtained in order to perform the measurements detailed below. Such images of dried drops are shown in FIG. 3.

Step c).

The third step of the process is a step of measuring at least one parameter indicative of the morphological features of the drop dried in step b), whereby a value for said parameter is obtained.

“Parameter indicative of the morphological features of the drop” means all parameters relating to the size and shape of the drop, and to the size and shape of the bead that is created on the periphery of the drop (see FIG. 3, OA). The parameters representative of the morphological features of the drop include the area of the drop, the surface profile (Z) of the drop, the height (H) of the bead and the width of the bead.

According to the invention, the parameter indicative of the morphological features of the dried drop is selected from (i) the maximum height (H) of the bead formed on the surface of the drop, (ii) the area of the drop and (iii) the surface profile (Z) of the drop.

It is understood that, in the process according to the invention, the skilled person may choose to measure a single parameter selected from (i) the maximum height (H) of the bead formed on the surface of the drop, (ii) the area of the drop and (iii) the surface profile (Z) of the drop, or measure the values of two parameters or measure the values of the three parameters mentioned above. All these parameters can be measured from 2D images and/or 3D images of the dried drop on a flat substrate, in particular a glass slide.

According to a particular implementation of the invention, the measurement step c) is carried out by a white-light interferometry method, to obtain 3D images of the dried drops.

Interferometry is a measurement method that exploits interference between several coherent waves.

White-light interferometers use special optical configurations and short coherence length light sources, which optimize the interaction between the light reflected from the measured object and the reference beam.

The measurement itself is based on the principle of the Michelson interferometer. The light is collimated from the light source and then divided into two beams: an object beam and a reference beam. The object beam is reflected by the measured object, and the reference beam is reflected on a reference mirror. The light reflected from each beam is captured and recombined at the beam splitter. The superimposed beams are then imaged by a camera.

Step d): Comparing Each Value Measured in Step c) with a Reference Value Representative of a Reference Synovial Fluid.

Each parameter value measured in step c) must be placed in perspective with a corresponding reference value, in order to be able to draw a diagnostic conclusion on the presence of joint disease in a mammal and/or on the type of joint disease present and/or to assess the severity of said joint disease.

Reference Value

For the purposes of the invention, “reference value” means a value of a parameter indicative of the morphological features of one or more drops of SF, representative of one or more reference synovial fluids.

“Reference synovial fluid” means, for the purposes of the invention, a sample of SF derived from a mammal whose status with respect to a given joint disease is known.

This may include samples of SF derived from mammals which are not affected by joint disease, or conversely which have a specific joint disease and whose stage is known.

According to one implementation of the invention, the reference value is selected from a value representative of a synovial fluid from a mammal not affected by joint disease, and a value representative of a synovial fluid from a mammal affected by joint disease at a given stage, in particular at a non-inflammatory stage.

It is in fact difficult to obtain synovial fluid samples from healthy individuals. Thus, reference samples are generally samples derived from mammals whose disease stage is known and characterized, for example whose disease stage is characterized as “non-inflammatory”.

It is understood that a reference synovial fluid will be derived from a mammal of the same species as the mammal whose status with respect to a given joint disease is tested. In particular, to diagnose joint disease in a human being, the reference SF will be derived from a human being.

It is also understood that a reference synovial fluid will be derived from a mammal whose status or type or severity stage with respect to a specific joint disease is known, to diagnose or determine the type or assess the severity of said joint disease. In particular, to diagnose or assess the severity of osteoarthritis, the reference SF will be derived from a mammal whose osteoarthritis status is known.

In some cases, several reference values may be used. For example, to assess the severity of osteoarthritis in a mammal, the value of an indicative parameter may be compared with a first reference value representative of an SF derived from a mammal with severe osteoarthritis, and with a second reference value representative of an SF derived from a mammal with moderate osteoarthritis.

It is also understood that for each parameter indicative of the morphological features of the dried drop, a reference value of said parameter measured from one or more reference synovial fluids will be assigned.

According to a preferred implementation of the process, said reference value is an average value measured from several SF samples derived from a plurality of mammals whose status with respect to a given joint disease is known, in particular from mammals which are not affected by joint disease, and in particular from healthy, i.e. joint disease free, dogs or humans.

In yet another embodiment, the reference value is a so-called “cut-off” value, which is determined from values determined from (i) SF samples from mammals with joint disease, and (ii) values determined from SF samples from healthy mammals, i.e. those not affected by the same joint disease (control SF).

An average reference value can be defined, this “cut-off” value clearly separating values obtained from control SF samples without joint disease from those of mammals with joint disease.

In this embodiment, a mammal tested according to the process of the invention will be considered to have joint disease when the value of the parameter measured from the dried drop of SF from said mammal is significantly different from the reference cut-off value.

Significant Differences with the Reference Values

Examples 1 and 2 presented in the present application were carried out on synovial fluid samples from healthy dogs and from dogs with signs of cartilage damage and varying degrees of inflammation. Example 3 relates to synovial fluid samples from individuals with osteoarthritis. Example 4 relates to synovial fluid samples from rabbits developing surgically induced osteoarthritis by surgical transection of the anterior cruciate ligament.

As is known to the skilled person, the results shown on these samples also apply to SF samples from other mammalian species and are therefore applicable to other mammalian species.

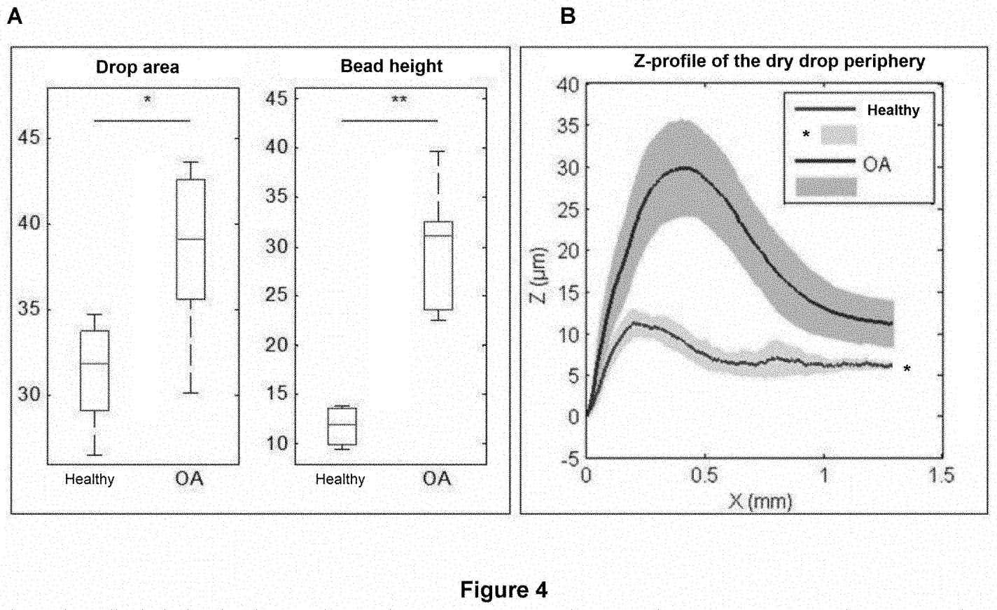

The morphological features of dried SF drops were observed on two-dimensional (2D) images, as well as on 3D images taken by white-light interferometry. The following results were observed:
the area of the drops is significantly larger in the sick (OA) dog group than in the healthy dog group (FIG. 4A);
the height of the bead formed at the periphery of the drop is significantly higher in the sick dog group (mean H=30.09 μm) than in the healthy dog group (mean H =11.81 μm) (FIG. 4A);
the value of the Z (Z/X) surface profile of the drop is much higher in the sick dog group than the reference value of this surface profile measured in the healthy dog group (FIG. 4B).
in the “rabbit” model, the drop area is significantly larger in the rabbit group with OA at an inflammatory stage (IS) than in the rabbit group with OA at a non-inflammatory stage (NIS) (FIG. 10A);
similarly, bead height is significantly higher when the synovial fluid comes from joints at an inflammatory stage (FIG. 10B);
the value of the Z (Z/X) surface profile of the drop is much higher for synovial fluid from joints at an inflammatory stage (FIG. 10C).

Thus, the following deductions can therefore be made:
a drop area value greater than a reference drop area value, representative of one or more reference synovial fluids from mammals not affected by joint disease, is indicative of the presence of joint disease;
a bead height value (H) greater than a reference bead height value (H_{R}), representative of one or more reference synovial fluids from mammals not affected by joint disease, is indicative of the presence of joint disease;
a drop surface profile value (Z) greater than a reference drop surface profile value (Z_{R}), representative of one or more reference synovial fluids from mammals not affected by joint disease, is indicative of the presence of joint disease;
the drop area, bead height and surface profile values are indicative of an inflammatory stage of joint disease.

Additional Steps

Raman spectroscopy, or Raman spectrometry, is a non-destructive method of observing and characterizing the molecular composition and external structure of a material, which exploits the physical phenomenon whereby a medium slightly changes the frequency of the light flowing through it. This frequency shift, known as the “Raman effect”, corresponds to an energy exchange between the light beam and the medium, and gives information on the composition of the medium itself.

Raman spectroscopy is performed by sending monochromatic light onto a sample and analyzing the scattered light. The information obtained by measuring and analyzing this shift makes it possible to define certain properties of the medium.

Thus, Raman spectroscopy of the dried SF drop provides information on the chemical composition and in particular on the presence of proteins contained in said drop.

According to a preferred implementation of the invention, the in vitro process for diagnosing joint disease in a mammal and/or determining the type of joint disease and/or evaluating the severity of said disease and/or predicting the course of said disease, further comprises the following steps:

e) determining the Raman spectrum of the drop dried in step b), and

f) comparing the Raman spectrum determined in step e) with a reference Raman spectrum representative of a reference synovial fluid.

As shown in the examples, all Raman spectra of SF from healthy dogs show only very small differences, as evidenced by the small standard deviation. Thus, a reference Raman spectrum can in particular be an average of several Raman spectra representative of several synovial fluid samples, from healthy dogs.

As presented in Example 2, it was found that the shape of the protein structure bands, as well as their intensities, vary significantly between the Raman spectra of SF samples from healthy dogs, and those from dogs with joint disease.

In particular, in the Raman spectra of SF samples from dogs with joint disease:
new bands appear at locations 970, 1248, 1575 cm⁻¹ and 1542 cm⁻¹, attributed respectively to fibrin and hemoglobin, resulting from inflammation of the joint (Virkler and Lednev, 2010) (FIG. 5B).
The 910-990 cm⁻¹ region was found to be drastically different between healthy and diseased spectra: while only the 945 cm⁻¹ hyaluronic acid band is predominant on the healthy spectra, 960 cm⁻¹ and 970 cm⁻¹ bands appear on the spectra of osteoarthritis subj ects.
The 1020-1145 cm⁻¹ region is rich in information because it contains the bands corresponding to phenylalanine (1031 cm⁻¹), hyaluronic acid (1046, 1102 and 1127 cm⁻¹), chondroitin 6-sulfate (C6S) (1062 cm⁻¹) and protein backbone (1081 cm⁻¹).
Finally, the 1220-1280 cm⁻¹ amide III region is the third to be notably different. (FIG. 5C).

In conclusion, it turns out that all bands related to HA, C6S and the protein backbone decrease in SF samples from diseased dogs, while an increase is observed in bands of CH₂/CH₃ and amino acids, as well as bone mineral phosphate.

In general, the Raman spectrum of SF samples from diseased dogs is significantly different from that of reference samples (FIG. 6).

In addition, and as shown in Example 3, the drop bead height value (H) can be correlated with the presence of some Raman bands of SF drops, such as the 1448/1102; 1654/1102; and 1448/1317 cm⁻¹ bands, on both dog and human SF samples (see Table 4 and FIG. 8).

Applications of the Process According to the Invention

The process according to the invention for diagnosing and/or determining the type and/or assessing the severity of joint disease is particularly suitable for the following joint diseases: osteoarthritis, arthritis, rheumatoid arthritis, ankylosing spondylitis and arthralgia.

According to a preferred implementation of the invention, this process is suitable for diagnosing and/or determining the type and/or assessing the severity of osteoarthritis.

The severity of the joint disease may in particular be determined on the basis of previously defined clinical or biological scores, in particular those related to significant clinical symptoms and/or low cartilage mass and/or synovitis and/or bone remodeling.

According to one implementation of the invention, the severity/stage of joint disease corresponds to significant clinical symptoms and/or low cartilage mass and/or synovitis and/or bone remodeling.

The present invention also relates to an in vitro process for monitoring a mammal with joint disease, comprising the following steps:

a) depositing a drop of a synovial fluid sample from said mammal, obtained at a time T₁, onto a flat substrate made of an inorganic material, such as glass;

b) drying the drop deposited in step a);

c) measuring at least one parameter indicative of the morphological features of the drop dried in step b), whereby a value for said parameter is obtained, and

d) comparing each value measured in step c) with a value obtained by applying steps (a) to (c) of the process to a synovial fluid sample from said mammal obtained at a time T₀ prior to time T₁,

characterized in that said parameter is selected from (i) the maximum height (H) of the bead formed on the surface of the drop, (ii) the area of the drop and (iii) the surface profile (Z) of the drop.

According to one implementation of this process, no reference value is used since the two compared values are those obtained from SF samples from the same mammal, but at two different times.

According to a particular implementation of the process according to the invention, a reference value representative of a reference synovial fluid is used in comparison with the values obtained from the SF samples obtained at times T₀ and T₁.

Of course, in this process, it is also possible to compare a large number of values of a single parameter, obtained from SF samples obtained at times T₀, T₁, T_{z}, T₃ etc. and thus to follow the progression of the joint disease.

According to one implementation of this process, time T₀ corresponds to a time when the mammal with joint disease is not yet treated for said disease and corresponds in particular to a “before treatment” state.

According to another implementation of this process, time T₀ corresponds to a time when the mammal with joint disease begins treatment for said disease.

According to one implementation of this process, time T₁ corresponds to a time when the mammal with joint disease has been undergoing treatment for the disease for at least one day, two days, three days, one week, two weeks, three weeks, one month, two months, or at least three months.

According to another implementation of this process, time T₁ corresponds to a time when the mammal with joint disease has finished its treatment for said disease and corresponds in particular to an “after treatment” state.

This process makes it possible to monitor the course of a disease, and thus to classify the pathology into several categories such as: slow or rapid progression disease, degenerative disease, etc.

This process also makes it possible to evaluate the efficacy of a given treatment. In this case, said treatment will be considered effective if the value of (i) the height of the bead formed on the surface of the drop, and/or (ii) the area of the drop and/or (iii) the surface profile (Z) of the drop, as determined in step (a), is less than the corresponding reference value V₀ determined before the start of treatment at T₀.

According to a particular implementation of the invention, this process may be completed by the implementation of the following additional steps:

e) determining the Raman spectrum of the drop dried in step b), and

f) comparing the Raman spectrum determined in step e) with a reference Raman spectrum representative of a reference synovial fluid.

The present invention also relates to an in vitro process for determining the efficacy of a treatment for joint disease in a mammal with said disease, to which said treatment is administered, comprising the following steps:

a) depositing a drop of a synovial fluid sample from said mammal, obtained after the start of said treatment, onto a flat substrate made of an inorganic material, such as glass;

b) drying the drop deposited in step a);

c) measuring at least one parameter indicative of the morphological features of the drop dried in step b), whereby a value for said parameter is obtained, and

d) comparing each value measured in step c) with a reference value representative of a reference synovial fluid,

characterized in that said parameter is selected from (i) the maximum height (H) of the bead formed on the surface of the drop, (ii) the area of the drop and (iii) the surface profile (Z) of the drop.

Such a process for monitoring the course and/or efficacy of a treatment corresponds to what can be called a “companion test” which makes it possible to adapt or change a treatment, based on the specific response of a given individual to this treatment.

In this process, the reference value may in particular be representative of the synovial fluid of said mammal, obtained at a time T₀ before the start of treatment.

In this case, said treatment will be considered effective if the value of (i) the height of the bead formed on the surface of the drop, and/or (ii) the area of the drop and/or (iii) the surface profile (Z) of the drop, as determined in step (a), is less than the corresponding reference value V₀ determined before the start of treatment at T₀.

The reference value may also be representative of a reference synovial fluid obtained from a mammal with no joint disease.

In this case, said treatment will be considered effective if the value of (i) the height of the bead formed on the surface of the drop, and/or (ii) the area of the drop and/or (iii) the surface profile (Z) of the drop, as determined in step a), is substantially equal to the reference value.

Two reference values can also be used:
a reference value representative of the synovial fluid of said mammal, obtained at a time T₀ before the start of treatment; and
a reference value representative of a reference synovial fluid obtained from a mammal with no joint disease.

In this case, said treatment will be considered effective if the value of (i) the height of the bead formed on the surface of the drop, and/or (ii) the area of the drop and/or (iii) the surface profile (Z) of the drop, as determined in step (a), approaches the reference value representative of a reference synovial fluid while deviating from the value V₀ determined before the start of treatment at T₀.

According to a preferred implementation of the invention, this process may be completed by the implementation of the following additional steps:

e) determining the Raman spectrum of the drop dried in step b), and

f) comparing the Raman spectrum determined in step e) with a reference Raman spectrum representative of a reference synovial fluid.

The present invention also relates to a process for screening a non-human mammal for candidate compounds intended to treat at least one joint disease, comprising the following steps:

a) depositing a drop of a synovial fluid sample from said mammal, obtained after the start of administration of one of said candidate compounds to said non-human mammal, onto a flat substrate made of an inorganic material, such as glass;

b) drying the drop deposited in step a);

c) measuring at least one parameter indicative of the morphological features of the drop dried in step b), whereby a value for said parameter is obtained, and

d) comparing each value measured in step c) with a reference value representative of a reference synovial fluid

characterized in that said parameter is selected from (i) the maximum height (H) of the bead formed on the surface of the drop, (ii) the area of the drop and (iii) the surface profile (Z) of the drop.

In this screening process, the reference value may in particular be representative of the synovial fluid of said non-human mammal, obtained at a time T₀ before the start of administration of a candidate compound.

In this case, said candidate compound will be considered effective if the value of (i) the height of the bead formed on the surface of the drop, and/or (ii) the area of the drop and/or (iii) the surface profile (Z) of the drop, as determined in step a), is less than the corresponding reference value V₀ determined before the start of administration at T₀.

The reference value may also be representative of a reference synovial fluid obtained from a mammal with no joint disease.

In this case, said candidate compound will be considered effective if the value of (i) the height of the bead formed on the surface of the drop, and/or (ii) the area of the drop and/or (iii) the surface profile (Z) of the drop, as determined in step a), is substantially equal to the reference value.

Two reference values can also be used:
a reference value representative of the synovial fluid of said mammal, obtained at a time T₀ before the start of administration of the candidate compound; and
a reference value representative of a reference synovial fluid obtained from a mammal with no joint disease.

In this case, said candidate compound will be considered effective if the value of (i) the height of the bead formed on the surface of the drop, and/or (ii) the area of the drop and/or (iii) the surface profile (Z) of the drop, as determined in step (a), approaches the reference value representative of a reference synovial fluid while deviating from the value V₀ determined before the start of administration at T₀.

According to a preferred implementation of the invention, this process may be completed by the implementation of the following additional steps:

e) determining the Raman spectrum of the drop dried in step b), and

f) comparing the Raman spectrum determined in step e) with a reference Raman spectrum representative of a reference synovial fluid.

In particular, owing to this in vivo screening process, the following compounds with anti-osteoarthritis activity can be tested: chondroitin sulfate, glucosamine, diacerein, avocado and soya unsaponifiables, and hyaluronic acid.

Naturally, this process is mainly intended to test new compounds whose anti-osteoarthritis activity has not yet been demonstrated.

Processes for Molecular Characterization of a Mammalian Synovial Fluid

The present invention also concerns a process for the molecular characterization of a mammalian synovial fluid, comprising the following steps:

a. depositing a drop of a synovial fluid sample from said mammal onto a flat substrate made of an inorganic material, such as glass;

b. drying the drop deposited in step a);

c. measuring:
the area of the drop dried in step b), whereby a value (A) for this area is obtained, and/or
the maximum height (H) of the bead formed on the surface of the drop dried in step b), whereby a value (H) for said height is obtained, and
d. comparing these values (A) and/or (H) with the following ranges of values:
   the value (A) is compared with a range of values correlated with the concentration of hyaluronic acid present in a synovial fluid; and
   the value (H) is compared with a range of values correlated with the concentration of proteins present in a synovial fluid.

As presented in Example 2, based on the results presented in Table 3 and FIG. 9, a very clear correlation (R²>70%) is observed between the drop area value (A) and the concentration of hyaluronic acid in the synovial fluid; as the drop area increases, the concentration of hyaluronic acid in the synovial fluid decreases.

Thus, a range of drop area values, including values Aᵢ with i=1 to n, n being at least equal to 5, preferentially equal to or greater than 10, can be indexed to a range of hyaluronic acid concentrations, as follows:

Aᵢ corresponds to a hyaluronic acid concentration Cᵢ;

Aᵢ₊₁ corresponds to a hyaluronic acid concentration Cᵢ₊₁;

Aᵢ₊₂ corresponds to a hyaluronic acid concentration Cᵢ₊₂;

Aₙ corresponds to a hyaluronic acid concentration Cₙ.

Such a range of values will be easily obtained by a skilled person, by applying conventional hyaluronic acid concentration measurement techniques.

This correlation makes it possible to carry out a molecular characterization of a synovial fluid, in particular to determine its hyaluronic acid concentration, from the value of the area of the dried drop of said synovial fluid, without further handling.

Thus, by simply measuring the area (A) of a dried drop, it is possible to determine the hyaluronic acid level in a synovial fluid sample, without the need for more sophisticated and expensive analytical techniques.

Similarly, as illustrated in Table 4 and FIG. 8, a very clear correlation (R²>85%) is observed between the bead height value (H) and the protein concentration in the synovial fluid; the higher the bead height, the higher the protein concentration in this synovial fluid.

Thus, a range of bead height values, including values Hᵢ with i=1 to n, n being at least equal to 5, preferentially equal to or greater than 10, can be indexed to a range of protein concentrations, as follows:

Hᵢ corresponds to a protein concentration Cpᵢ;

Hᵢ₊₁ corresponds to a protein concentration Cpᵢ₊₁;

Hᵢ₊₂ corresponds to a protein concentration Cpᵢ₊₂;

Hₙ corresponds to a protein concentration Cpₙ

Such a range of values will be easily obtained by a skilled person by applying conventional protein content determination techniques.

This correlation makes it possible to carry out a molecular characterization of a synovial fluid, in particular to determine its protein concentration, from the height value of the bead formed on the surface of the dried drop of said synovial fluid, without further handling.

Thus, by simply measuring the height (H) of the bead on the surface of a dried drop, it is possible to determine the protein content of a synovial fluid sample without the need for more sophisticated and expensive analytical techniques.

The two parameters drop area (A) and bead height (H) can advantageously be measured together on the same synovial fluid sample, to determine the hyaluronic acid and protein concentration of this synovial fluid.

The skilled person will thus be able to obtain information on the type of joint disease, or the stage of the joint disease, based on the hyaluronic acid and protein concentrations as determined according to the process of the invention.

In particular, if these measurements are made on synovial fluid samples collected at different points in time, it will be possible for the skilled person to determine the progression of the disease in a patient, based on the change over time in the hyaluronic acid and protein concentration in the synovial fluid.

### EXAMPLES

The examples presented below are for illustrative purposes only and in no way limit the invention described in the present application.

Materials and Methods

Synovial Fluid (SF) Samples

SF samples are collected in vivo from healthy and arthritic dogs. The sampling protocol, in accordance with European Community legislation, has been validated by the Ethics Committee (VetAgro Sup, referral no. 1187 for healthy SF, no. 1408 for pathological SF).

The SF samples were placed in sealed Eppendorf tubes, without additives, then stored at −80° C. until use.

Healthy SF samples were obtained by arthrocentesis (20 G needle, 5 ml syringe) in six adult Beagle dogs, aged 2 to 3 years, clinically and radiologically healthy after intramuscular sedation and surgical preparation of the joints. All samples were free of blood contamination and of normal appearance (transparency, turbidity, viscosity, color).

Pathological SF samples were collected sterile in the operating room from dogs of clients of the CHEV clinic at the VetAgro Sup veterinary campus and requiring surgery under general anesthesia. The different joints operated on showed signs of cartilage damage and varying degrees of inflammation assessed by the surgeons and noted on each operative report.

Table 1 below summarizes the characteristics of the different samples:

TABLE 1

| | Age | Weight | | |
|---|---|---|---|---|
| SF | (months) | (kg) | Deg | Clinical diagnosis |
| H1 | 34 | 10.6 | | (Healthy) |
| H2 | 35 | 8.8 | | (Healthy) |
| H2 | 34 | 9.8 | | (Healthy) |
| H4 | 35 | 11.5 | | (Healthy) |
| H5 | 34 | 11.0 | | (Healthy) |
| H6 | 11 | 12.8 | | (Healthy) |
| OA1 | 91 | 56.2 | J | Ruptured cruciate ligaments |
| OA2 | 56 | 52.7 | J | Ruptured cruciate ligaments |
| OA3 | 29 | 40.5 | J | Ruptured cruciate ligaments |
| OA4 | 60 | 28 | J | Ruptured cruciate ligaments |
| OA5 | 45 | 0 | J | Ruptured cruciate ligaments |
| OA6 | 29 | 58.5 | + | Severe post TPLO |

Drop Deposition

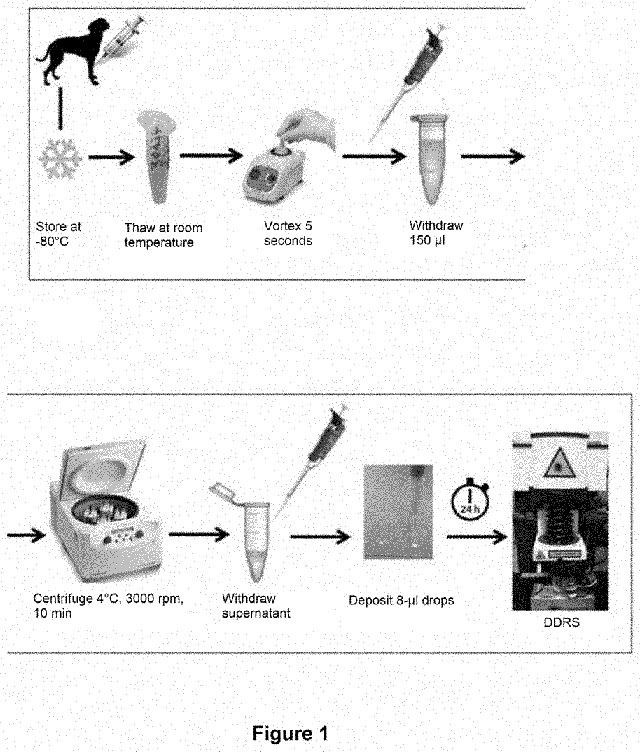

After thawing at room temperature, 150 μl of sample is centrifuged at 3000 rpm (1000 g) for 10 min at 4° C. The pellet is separated from the supernatant. Glass slides are first degreased with 70° alcohol. Supernatant drops (8 μl) are then placed on the slides and allowed to dry semi-covered at room temperature for one day (FIG. 1).

Observation of 2D and 3D Photos of Dried Drops

A standard microscope equipped with a 2048×2048 pixel camera was used to photograph the dried drops, with a 2.5× objective. Depending on the drop size, several photos may have been necessary. The ‘Image J’ software was used to reconstruct 2D photos using the ‘MosaicJ’ plugin and to measure the area of each drop.

3D topographies were obtained by white-light interferometry (smartWLl-microscope, GBS mbH, Germany) on the periphery of each drop (right side) in vertical scanning interferometry (VSI) mode with a Michelson objective.

The MountainsMap® analysis software (DigitalSurf, France) was used to reconstruct topographic maps measuring 1.4×1.07 mm and to record associated data (X, Y, Z).

MATLAB® (The MathWorks, MA, USA, version R2013a) routines were developed specifically to calculate the Z profiles along each topographic map.

Drop Deposition RS (DDRS)

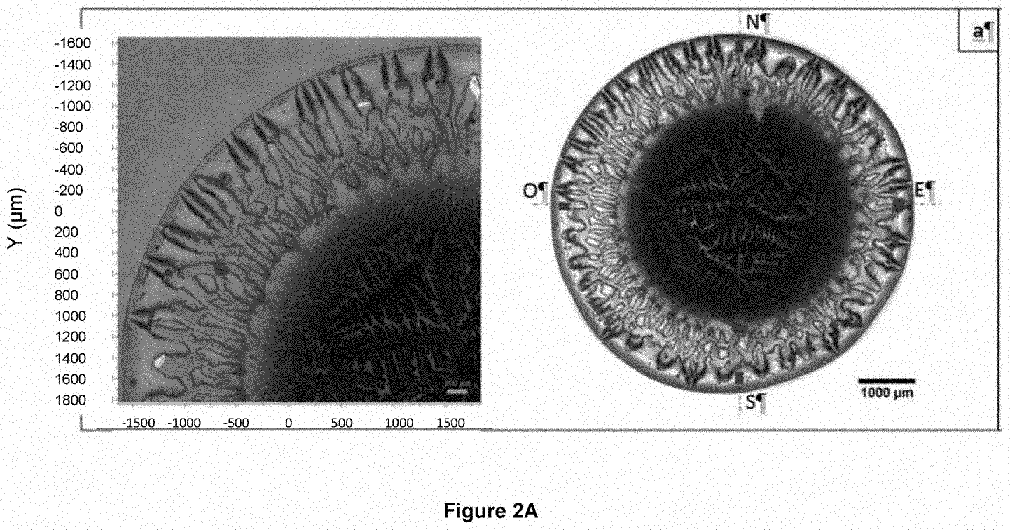

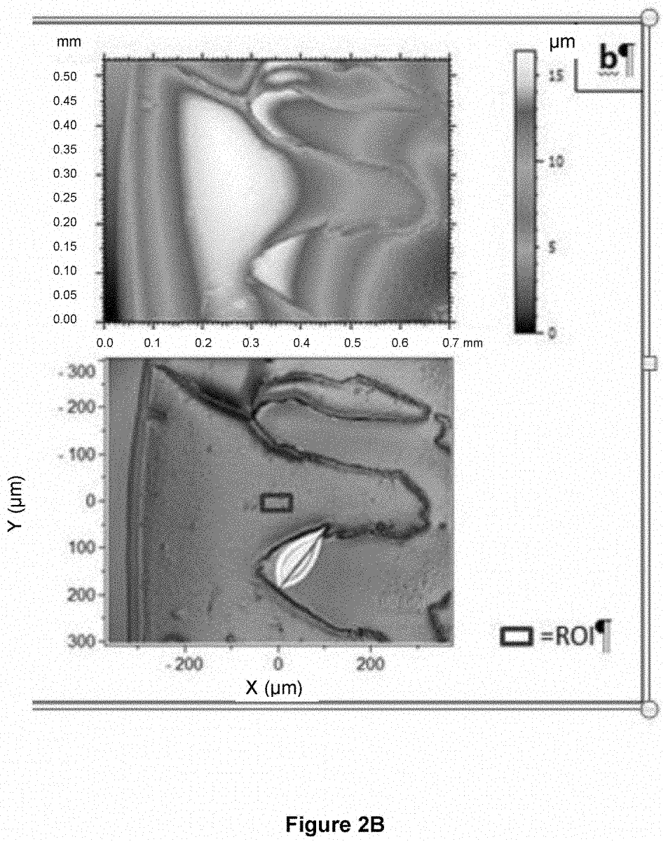

Spectral acquisitions were made on a confocal Raman microscope (LabRAM HR800®, Horiba Jobin Yvon, Villeneuve d′Ascq, France). Four regions of interest (ROIs) were selected with a 10× objective at the four cardinal points of each drop, measuring 20×60 μm², in the peripheral area where the pre-concentration of solutes is maximum, as was verified by interferometry (FIG. 2).

Before any acquisition, the system was calibrated using the 520.7 cm⁻¹ line of a reference silicon sample. Spectral acquisitions were made with a 50× objective, numerical aperture 0.75 and a laser wavelength at 632.8 nm (HeNe, sample power 12 mW) giving a spot size of 1 μm. Raman scattering was measured by a CCD detector (1024×256 pixels cooled by Peltier effect at −70° C.). The spectral resolution is less than 1 cm⁻¹ thanks to the use of an 1800 lines/mm grating. The size of the confocal hole is adjusted to 200 μm for an axial resolution of 2 μm.

In each of the ROIs, six points were mapped, each spaced 20 μm from the other, in the spectral range 800 to 1780 cm⁻¹, with an acquisition time of 30 s and 3 accumulations. The spectra were recorded with the LabSpec6 software (Horiba Jobin Yvon, Villeneuve d'Ascq, France).

Preprocessing of Raman Results

All spectra had a high signal-to-noise ratio. All were preprocessed in MATLAB® (MathWorks, MA, USA, version R2013a) by developing user routines to eliminate fluorescence and remove the baseline.

For the comparison of spectral signatures and identification of Raman bands, the average spectrum of each SF drop was defined as the average of the average spectra of each ROI.

The 1002 cm⁻¹ phenylalanine band (called the bead breathing band) was used to normalize intensities (Esmonde-White et al., 2009), this band being not sensitive to protein conformational changes).

Gauss and Lorentz functions were used to fit peaks in specific bands. Conventionally in Raman spectroscopy, peak intensity ratios are used rather than the intensities themselves to detect differences between samples (Nyman et al., 2011), the ratios associated with fitted peaks were therefore calculated.

Statistical Analyses

All statistical tests were performed in MATLAB with the “Statistics and Machine Learning Toolbox”. Mann-Whitney tests were performed on all calculated ratios to verify statistical differences between healthy and osteoarthritic SF. The significance level was a risk −α of 5% (p<0.05). Pearson correlation tests were performed on these same ratios to find out if there are correlations between the ratios and the morphological characterizations of the drops.

Principal component analyses (PCAs) were also conducted on these ratios. PCA is a multivariate statistical method conventionally used on hyperspectral data to reduce the large number of variables contained in the spectra. The principal components (PCs) are calculated as a new set of decorrelated variables, linear combinations of the correlated initial variables, and explaining the maximum possible variance between the data. Scatter plots allow an easy visualization of the PCA results, each spectrum being represented by a point in the PC axis system.

### Example 1. Results Obtained by White-Light Interferometry

SF drops were deposited onto standard glass slides and 2D images of the dried drops were captured (FIG. 3).

Drop area was significantly larger (p=0.026) in the sick OA group (n=6) with a mean area (standard deviation) of 38.37 mm² (4.80 mm²) than in the healthy group (n=6) with a mean (standard deviation) of 31.30 mm² (2.85 mm²) (FIG. 4).

These microscope images revealed the presence of a black bead between the outer periphery and the crystalline center only visible on drops from the healthy group while a bead is clearly visible on the drops from the OA group.

The 3D interferometric images (FIG. 3) confirmed that the peripheral bead height is significantly higher (p=0.002) in the OA group (n=6) with a mean height (standard deviation) of 30.09 μm (5.80 μm), almost three times higher than that of the healthy group (n=6) with a mean height (standard deviation) of 11.81 μm (1.71 μm) (FIG. 4).

### Example 2. Results Obtained by Raman Spectral Analysis

No significant difference was observed between the different spectra of healthy SF as evidenced by the small standard deviation. The mean of the 6 spectra of healthy SF was then used to model the spectral signature of healthy SF (FIG. 5A).

Raman bands were identified and assigned from a literature review on synovial fluid, proteins, amino acids, blood and serum (Table 2).

To compare healthy and OA spectra, the main bands were identified, deconvoluted and the related ratios calculated. Thus, 21 peaks were selected, identified in Table 2, resulting in the calculation of 210 ratios (only half of the ratio matrix was used, considering that it was not necessary to calculate a ratio and its inverse).

TABLE 2
Raman bands of dried SF drops

| | | Raman |
|---|---|---|
| Raman | | peaks |
| band | | used |
| ~(cm⁻¹) | Assigned molecule | (n = 21) |
| 828 | Tyrosine doublet | |
| 852 | Tyrosine doublet | ✓ |
| 880 | δ(C—C) Tryptophan | |
| 896 | Hyaluronic acid | ✓ |
| 945 | Hyaluronic acid | ✓ |
| 960 | ν(PO₄³⁻) of phosphate bone | ✓ |
| 970 | Fibrin of blood | ✓ |
| 1002 | C—C aromatic bead of phenylalanine (breathing mode) | ✓ |
| 1031 | δ(C—H) of phenylalanine | ✓ |
| 1046 | Hyaluronic acid | ✓ |
| 1062 | ν(OSO₃⁻) of chondroitin 6-sulfate | ✓ |
| 1081 | ν(C—C) or ν(C—O) or ν(C—N) protein and lipids | ✓ |
| 1102 | Hyaluronic acid | ✓ |
| 1127 | Hyaluronic acid | ✓ |
| 1172 | Tyrosine | ✓ |
| 1207 | Tyrosine, phenylalanine | ✓ |
| 1242 | ν(C—N), δ(N—H) of Amide III (β-sheet conformation) | ✓ |
| 1248 | Fibrin of Blood | |
| 1275 | ν(C—N), δ(N—H) of Amide III (α-helix conformation) | ✓ |
| 1317 | CH₂ CH₃ twist of proteins | ✓ |
| 1339 | Tryptophan, CH₂ CH₃ wag of proteins | ✓ |
| 1363 | Heme of blood | |
| 1448 | δCH₂, δCH₃ in collagen | ✓ |
| 1542 | Heme of blood | |
| 1554 | Tryptophan | |
| 1575 | Fibrin of blood | |
| 1586 | Phenylalanine | |
| 1605 | Phenylalanine | |
| 1615 | Tyrosine | |
| 1654 | ν(C═O) stretching of Amide I (α helix conformation) | ✓ |
| 1670 | ν(C═O) stretching of Amide I (β sheet conformation) | ✓ |

The assignments were made from the bibliography on SF, proteins, amino acids, hyaluronic acid, blood and serum (Alkrad et al., 2003; Bansil et al., 1978; Dingari et al., 2012; Ellis et al., 2009; Esmonde-White et al., 2008; Mandair et al., 2006; Rygula et al., 2013; Tuma, 2005; Virkler and Lednev, 2010; Wei et al., 2008)

The structural bands of proteins, such as amide III between 1242 and 1275 cm⁻¹ and amide I between 1654 and 1670 cm⁻¹ are particularly recognizable, as well as amino acid bands such as phenylalanine (“breathing” mode of the aromatic bead at 1002 cm⁻¹) and tyrosine (doublet at 828 and 850 cm⁻¹). The protein content can also be identified by the different modes of CH₂CH₃ at 1448 cm⁻¹ (“bending” mode), 1317 cm⁻¹ (“twisting” mode) and 1339 cm⁻¹ (“wagging” mode). The presence of hyaluronic acid (HA) is identifiable by the 945 cm⁻¹ band and its contribution in the 1020-1140 cm⁻¹ region (Esmonde-White et al., 2008).

The shape of the bands and their intensities vary significantly between the healthy and OA spectra, with the appearance of new bands such as 970, 1248 and 1575 cm⁻¹ and at 1542 cm⁻¹ attributed respectively to fibrin and hemoglobin, resulting from inflammation of the joint (Virkler and Lednev, 2010) (FIG. 5B).

Several spectral regions of interest have attracted attention (FIG. 5C). The 910-990 cm⁻¹ region was found to be drastically different between healthy and OA spectra: while only the HA band at 945 cm⁻¹ is predominant on healthy spectra, bands at 960 cm⁻¹ and 970 cm⁻¹ appear on the OA spectra. The 1020-1145 cm⁻¹ region is rich in information because it contains phenylalanine (1031 cm⁻¹), HA (1046, 1102 and 1127 cm⁻¹), chondroitin 6-sulfate (C6S) (1062 cm⁻¹) bands and the protein backbone (1081 cm⁻¹). Finally, the 1220-1280 cm⁻¹ amide III region is the third to be notably different.

Univariate Statistical Analyses

Univariate Mann-Whitney tests were performed on the 210 ratios calculated to compare the healthy group (n=6) and the OA group (n=6). One hundred and sixty-four of them were found to be significantly different. Analysis of these significantly different ratios leads to the conclusion that all bands related to HA, C6S and the protein backbone are decreasing while an increase is observed in bands of CH₂/CH₃ and amino acids, as well as bone mineral phosphate.

Linear Regressions

Pearson correlation tests were carried out on the 210 ratios to find possible correlations between the ratios and the morphological characterizations of the drops (bead height and drop area).

The tests showed that 4 ratios had a correlation greater than 70% (p<10-³) with drop area and 18 ratios had a correlation greater than 80% (p<10-⁴), including 8 with a correlation greater than 85%, with bead height. All these ratios were part of the 164 significantly different ratios between healthy and OA groups (Tables 3 and 4).

TABLE 3
List of the four ratios related to hyaluronic acid, showing a high
degree of correlation with drop area: means and standard deviations
of the ratios, p-value of the Mann-Whitney tests, change trend, drop
area correlation coefficients.

| | | | | | Mann- | | Area |
|---|---|---|---|---|---|---|---|
| | Healthy | | OA | | Whitney | | correlation |
| Ratios | Mean | SD | Mean | SD | p | Trend | R² (%) |
| 1317/896 cm⁻¹ | 3.00 | 0.10 | 3.68 | 0.31 | 0.004 | [symbol] | 77.3 |
| 1339/896 cm⁻¹ | 2.74 | 0.06 | 3.65 | 0.29 | 0.002 | [symbol] | 72.6 |
| 1317/945 cm⁻¹ | 1.30 | 0.04 | 1.86 | 0.26 | 0.002 | [symbol] | 70.8 |
| 1062/1046 cm⁻¹ | 0.88 | 0.06 | 1.05 | 0.05 | 0.002 | [symbol] | 71.8 |

FIG. 9 graphically shows this very clear correlation (R²>70%) between the area value of the drops and the concentration of hyaluronic acid in the synovial fluid; as the drop area increases, the concentration of hyaluronic acid in the synovial fluid decreases.

Thus, a range of drop area values can be indexed to a range of hyaluronic acid concentrations.

This correlation makes it possible to carry out a molecular characterization of a synovial fluid, in particular to determine its hyaluronic acid concentration, from the value of the area of the dried drop of said synovial fluid, without further handling.

Another important correlation was shown between the height (H) of the bead present on the surface of the drops and the level of proteins present in the SF tested, as shown in Table 4 below and FIG. 8.

TABLE 4
List of eight ratios showing a high degree of correlation with bead
height: means and standard deviations of the ratios, p-value of
the Mann-Whitney tests, change trend, bead height correlation
coefficients.

| | | | | | Mann- | | Height |
|---|---|---|---|---|---|---|---|
| | Healthy | | OA | | Whitney | | correlation |
| Ratios | Mean | SD | Mean | SD | p | Trend | R² (%) |
| 1102/852 cm⁻¹ | 0.92 | 0.06 | 0.42 | 0.07 | 0.002 | [symbol] | 88.3 |
| 1102/1002 cm⁻¹ | 0.20 | 0.02 | 0.10 | 0.01 | 0.002 | [symbol] | 86.0 |
| 1102/1031 cm⁻¹ | 0.84 | 0.04 | 0.53 | 0.07 | 0.002 | [symbol] | 90.5 |
| 1127/1102 cm⁻¹ | 1.28 | 0.07 | 1.97 | 0.30 | 0.002 | [symbol] | 88.2 |
| 1172/1102 cm⁻¹ | 0.38 | 0.05 | 0.93 | 0.12 | 0.002 | [symbol] | 87.1 |
| 1448/1102 cm⁻¹ | 2.51 | 0.21 | 6.04 | 0.92 | 0.002 | [symbol] | 86.6 |
| 1654/1102 cm⁻¹ | 3.30 | 0.32 | 6.46 | 0.90 | 0.002 | [symbol] | 86.3 |
| 1448/1317 cm⁻¹ | 1.37 | 0.03 | 1.70 | 0.07 | 0.002 | [symbol] | 88.6 |

FIG. 8 graphically shows this very clear correlation (R²>85% in dogs) between the height value of the beads, and the protein concentration in the synovial fluid; as bead height increases, the protein concentration in this synovial fluid increases.

Thus, a range of bead height values can be indexed to a range of protein concentrations.

This correlation makes it possible to carry out a molecular characterization of a synovial fluid, in particular to determine its protein concentration, from the height value of the bead formed on the surface of the dried drop of said synovial fluid, without further handling.

Multivariate Statistical Analysis

In order to evaluate the statistical differences between the spectra of healthy and OA SF, a PCA was performed using the 210 ratios as input variables. The scatter plot of the first four PCA scores, representing 92.2% of the total explained variance, showed that the OA and healthy groups are clearly separated (FIG. 6).

### Example 3. Analysis of Human Synovial Fluid Drops

In this study, human SF samples were provided “ready-to-use” by the University Hospital of Geneva (HUG). These were SF collected in vivo from patients of radiographic grades 2 and 4. These samples were provided with a certain number of patient data (age, BMI) and biomarkers such as leptin, interleukin-6 (IL6) assays and WOMAC pain and function scores.

These SF samples were processed as presented above for dog SF samples, i.e. drop deposition, 2D and 3D images of dried drops and Raman spectra.

Statistical Analyses

All statistical tests were performed in MATLAB® with the “Statistics and Machine Learning Toolbox”. Pearson correlation tests were performed on all ratios to determine the existence of correlations between the 210 ratios and (i) biomarkers and (ii) morphological features of the drops (bead height and drop area).

HUG SF/IL6 Assay Correlation

Correlation tests showed a linear correlation of more than 50% between three ratios, all related to chondroitin 6-sulfate and the Interleukin-6 assay (FIG. 7), taken as a log 10. These correlations seem very relevant since it is known that there is a relationship between chondroitin 6-sulfate (one of the glycosaminoglycans in the cartilage matrix) and Interleukin-6.

HUG SF+CN/Height Correlation

As for the dogs, Pearson correlation tests were performed on the 210 ratios to find significant correlations between the ratios and the morphological features of the drops (bead height and drop area). The tests showed in particular that the ratios strongly correlated with bead height in humans are also very strongly correlated in dogs (Table 5) and that the ratios are very close (FIG. 8), making these ratios very relevant.

TABLE 5
Three ratios show a correlation greater than 70% (p < 10−⁴) with bead
height of human SF drops. They are also significantly correlated with
bead heights of dog SF drops.

| | Height correlation | |
|---|---|---|
| Ratios | R² HUG (%) | R² Dogs (%) |
| 1448/1102 cm⁻¹ | 72.1 | 86.6 |
| 1654/1102 cm⁻¹ | 73.9 | 86.3 |
| 1448/1317 cm⁻¹ | 85.6 | 88.6 |

### Example 4. Analysis of Rabbit Synovial Fluid Drops

An animal model of surgically induced osteoarthritis has been developed in rabbits: this model is called the “anterior cruciate ligament transection (ACLT) model”. This model is particularly appropriate for studying the early stages of osteoarthritis (Madry et al., 2016).

Two groups of rabbits were studied:
A “2 weeks after ACLT induction” group corresponding to the inflammatory stage of the disease, hereinafter referred to as “Inflammatory stage (IS)”;
A “6 weeks after ACLT induction” group corresponding to a non-inflammatory stage of osteoarthritis, hereinafter referred to as the “Non-inflammatory stage (NIS)”.

In each group, half of the individuals were treated by injection of hyaluronic acid into the joint; however, no notable difference was observed between treated and untreated rabbits with respect to the parameters studied, thus these differences are not presented here.

The volume of synovial fluid collected is greater in the IS group than in the NIS group, due to post-surgical inflammation.

Joint tissue samples from the IS group do not yet show any visible macroscopic changes typical of osteoarthritis. Conversely, tissue samples from the NIS group show tissue damage characteristic of osteoarthritis. However, in the IS group, the viscoelastic properties of the joint are already affected.

The synovial fluid collected from these rabbits was studied as presented in the “Materials and Methods” section above.

Table 6 below and FIG. 10 present the drop area and bead height values of the identified synovial fluids.

TABLE 6
Mean and standard deviation values of SF drop area and bead height.

| | | Area | | Height | |
|---|---|---|---|---|---|
| | | (mm²) | | (mm) | |
| Groups | | Mean | σ (±) | Mean | σ (±) |
| IS | (n = 8) | 38.07 | 2.70 | 27.55 | 1.52 |
| NIS | (n = 9) | 29.29 | 3.21 | 21.74 | 4.58 |

| | | |
|---|---|---|
| p-value | 3 · 10⁻⁴ | 0.0037 |

Synovial fluid from IS rabbits has much higher drop area and bead height values than synovial fluid from NIS rabbits, from joints with tissue damage typical of osteoarthritis but where inflammation is reduced.

Thus, the parameters “drop area” and “bead height” can be used to determine whether the disease is at an inflammatory or non-inflammatory stage.

### REFERENCES

Alkrad, J. A., Mrestani, Y., Stroehl, D., Wartewig, S., and Neubert, R. (2003). Characterization of enzymatically digested hyaluronic acid using NMR, Raman, I R, and UV-Vis spectroscopies. J. Pharm. Biomed. Anal. 31, 545-550.
Bansil, R., Yannas, I. V., and Stanley, H. E. (1978). Raman spectroscopy: a structural probe of glycosaminoglycans. Biochim. Biophys. Acta 541, 535-542.
Beyer C, Zampetaki A, Lin N Y, Kleyer A, Perricone C, Iagnocco A, Distler A, Langley S R, Gelse K, Sesselmann S, Lorenzini R, Niemeier A, Swoboda B, Distler J H, Santer P, Egger G, Willeit J, Mayr M, Schett G, Kiechl S. (2015) Signature of circulating microRNAs in osteoarthritis. Ann Rheum Dis.; 74(3):e18.
Dingari, N. C., Horowitz, G. L., Kang, J. W., Dasari, R. R., and Barman, I. (2012). Raman Spectroscopy Provides a Powerful Diagnostic Tool for Accurate Determination of Albumin Glycation. PLoS ONE 7, e32406.
Ellis, R., Green, E., and Winlove, C. P. (2009). Structural Analysis of Glycosaminoglycans and Proteoglycans by Means of Raman Microspectrometry. Connect. Tissue Res. 50, 29-36.
Esmonde-White, K. A., Mandair, G. S., Raaii, F., Roessler, B. J., and Morris, M. D. (2008). Raman spectroscopy of dried synovial fluid droplets as a rapid diagnostic for knee joint damage. A. Mahadevan-Jansen, W. Petrich, R. R. Alfano, and A. Katz, eds. p. 68530Y.
Esmonde-White, K. A., Mandair, G. S., Esmonde-White, F. W., Raaii, F., Roessler, B. J., & Morris, M. D. (2009a). Osteoarthritis screening using Raman spectroscopy of dried human synovial fluid drops. In Proc. of SPIE Vol (Vol. 7166, pp. 71660J-1).
Esmonde-White, K. A., Mandair, G. S., Raaii, F., Jacobson, J. A., Miller, B. S., Urquhart, A. G., Roessler, B. J., and Morris, M. D. (2009b). Raman spectroscopy of synovial fluid as a tool for diagnosing osteoarthritis. J. Biomed. Opt. 14, 34013.
Esmonde-White K A, Esmonde-White F W, Morris M D, Roessler B J. (2014) Characterization of biofluids prepared by sessile drop formation. Analyst. June 7; 139(11):2734-41.
Lotz M, Martel-Pelletier J, Christiansen C, Brandi M L, Bruyere O, Chapurlat R, Collette J, Cooper C, Giacovelli G, Kanis J A, Karsdal M A, Kraus V, Lems W F, Meulenbelt I,
Pelletier J P, Raynauld J P, Reiter-Niesert S, Rizzoli R, Sandell L J, Van Spil W E, Reginster J Y. (2013) Value of biomarkers in osteoarthritis: current status and perspectives. Ann Rheum Dis. November; 72(11):1756-63.
Litwic A, Edwards M H, Dennison E M, Cooper C. (2013) Epidemiology and burden of osteoarthritis. Br Med Bull.; 105:185-99.
Madry, H., Kon, E., Condello, V., Peretti, G. M., Steinwachs, M., Seil, R., Berruto, M., Engebretsen, L., Filardo, G., and Angele, P. (2016). Early osteoarthritis of the knee. Knee Surg. Sports Traumatol. Arthrosc. 24, 1753-1762.
Mandair, G. S., Dehring, K. A., Roessler, B. J., and Morris, M. D. (2006). Detection of potential osteoarthritis biomarkers using surface enhanced Raman spectroscopy in the near-infrared. A. Mahadevan-Jansen, and W. H. Petrich, eds. p. 60930H.
Nyman, J. S., Makowski, A. J., Patil, C. A., Masui, T. P., O'Quinn, E. C., Bi, X., Guelcher, S. A., Nicollela, D. P., and Mahadevan-Jansen, A. (2011). Measuring Differences in Compositional Properties of Bone Tissue by Confocal Raman Spectroscopy. Calcif. Tissue Int. 89, 111-122.
Rygula, A., Majzner, K., Marzec, K. M., Kaczor, A., Pilarczyk, M., and Baranska, M. (2013). Raman spectroscopy of proteins: a review: Raman spectroscopy of proteins. J. Raman Spectrosc. 44, 1061-1076.
Tuma, R. (2005). Raman spectroscopy of proteins: from peptides to large assemblies. J. Raman Spectrosc. 36, 307-319.
Virkler, K., and Lednev, I. K. (2010). Raman spectroscopic signature of blood and its potential application to forensic body fluid identification. Anal. Bioanal. Chem. 396, 525-534.
Wei, F., Zhang, D., Halas, N. J., and Hartgerink, J. D. (2008). Aromatic Amino Acids Providing Characteristic Motifs in the Raman and SERS Spectroscopy of Peptides. J. Phys. Chem. B 112, 9158-9164.

1. In vitro process for diagnosing joint disease in a mammal and/or determining the type of joint disease and/or determining the stage of said disease and/or predicting the course of the disease, comprising the following steps:
a) depositing a drop of a synovial fluid sample from said mammal onto a flat substrate made of an inorganic material, such as glass;
b) drying the drop deposited in step a);
c) measuring at least one parameter indicative of the morphological features of the drop dried in step b), whereby a value for said parameter is obtained, and
d) comparing each value measured in step c) with a reference value representative of a reference synovial fluid,
wherein said parameter is selected from (i) the maximum height (H) of the bead formed on the surface of the drop, (ii) the area of the drop, and (iii) the surface profile (Z) of the drop.

2. The process according to claim 1, wherein the reference value is selected from a value representative of a synovial fluid from a mammal not affected by joint disease, and a value representative of a synovial fluid from a mammal affected by joint disease at a given stage, in particular at a non-inflammatory stage.

3. The process according to claim 1, wherein the process further comprises the following steps:
e) determining the Raman spectrum of the drop dried in step b), and
f) comparing the Raman spectrum determined in step e) with a reference Raman spectrum representative of a reference synovial fluid.

4. The process according to claim 1, wherein the measurement step c) is carried out by a white-light interferometry method.

5. The process according to claim 1, wherein the joint disease is osteoarthritis.

6. The process according to claim 1, wherein the stage of the joint disease corresponds to significant clinical symptoms and/or low cartilage mass and/or synovitis and/or bone remodeling.

7. In vitro process for monitoring a mammal with joint disease, comprising the following steps:
a) depositing a drop of a synovial fluid sample from said mammal, obtained at a time T₁, onto a flat substrate made of an inorganic material, such as glass;
b) drying the drop deposited in step a);
c) measuring at least one parameter indicative of the morphological features of the drop dried in step b), whereby a value for said parameter is obtained; and
d) comparing each value measured in step c) with a value obtained by applying steps (a) to (c) of the process to a synovial fluid sample from said mammal obtained at a time T₀ prior to time T₁,
wherein said parameter is selected from (i) the maximum height (H) of the bead formed on the surface of the drop, (ii) the area of the drop, and (iii) the surface profile (Z) of the drop.

8. In vitro process for determining the efficacy of a treatment for joint disease in a mammal with said disease to which said treatment is administered, comprising the following steps:
a) depositing a drop of a synovial fluid sample from said mammal, obtained after the start of said treatment, onto a flat substrate made of an inorganic material, such as glass;
b) drying the drop deposited in step a);
c) measuring at least one parameter indicative of the morphological features of the drop dried in step b), whereby a value for said parameter is obtained; and
d) comparing each value measured in step c) with a reference value representative of a reference synovial fluid,
wherein said parameter is selected from (i) the maximum height (H) of the bead formed on the surface of the drop, (ii) the area of the drop, and (iii) the surface profile (Z) of the drop.

9. Process for screening a non-human mammal for candidate compounds to treat at least one joint disease, comprising the following steps:
a) depositing a drop of a synovial fluid sample from said mammal, obtained after the start of administration of one of said candidate compounds to said non-human mammal, onto a flat substrate made of an inorganic material, such as glass;
b) drying the drop deposited in step a);
c) measuring at least one parameter indicative of the morphological features of the drop dried in step b), whereby a value for said parameter is obtained; and
d) comparing each value measured in step c) with a reference value representative of a reference synovial fluid,
wherein said parameter is selected from (i) the maximum height (H) of the bead formed on the surface of the drop, (ii) the area of the drop, and (iii) the surface profile (Z) of the drop.

10. Process for the molecular characterization of a mammalian synovial fluid, comprising the following steps:
a. depositing a drop of a synovial fluid sample from said mammal onto a flat substrate made of an inorganic material, such as glass;
b. drying the drop deposited in step a);
c. measuring:
the area of the drop dried in step b), whereby a value (A) for this area is obtained, and/or
the maximum height (H) of the bead formed on the surface of the drop dried in step b), whereby a value (H) for said height is obtained; and
d. comparing these values (A) and/or (H) with the following ranges of values:
the value (A) is compared with a range of values correlated with the concentration of hyaluronic acid present in a synovial fluid; and
the value (H) is compared with a range of values correlated with the concentration of proteins present in a synovial fluid.

11. The process according to claim 2, wherein the process further comprises the following steps:
e) determining the Raman spectrum of the drop dried in step b), and
f) comparing the Raman spectrum determined in step e) with a reference Raman spectrum representative of a reference synovial fluid.
